(19) United States
(12) Patent Application Publication
(10) Pub. No.: US 2007/0218084 A1
(43) Pub. Date: Sep. 20, 2007

(54) Method for the Mapping of the Epileptogenic Focus in the Pre-Surgical Evaluation of Patients with Intractable Epilepsy

(75) Inventors: Matteo Caleo, Montignoso (Massa Carrara) (IT); Yuri Bozzi, Pisa (IT); Laura Costantin, Belluno (IT); Cristina Richichi, Milano (IT); Alessandro Viegi, Asciano Pisano (Pisa) (IT); Flavia Antonucci, Pisa (IT); Marcella Funicello, Montecorice (Salerno) (IT); Marco Gobbi, Milano (IT); Tiziana Mennini, Milano (IT); Ornella Rossetto, Padova (IT); Cesare Montecucco, Padova (IT); Lamberto Maffei, Asciano Pisano (Pisa) (IT); Annamaria Vezzani, Milano (IT)

Correspondence Address: AKERMAN SENTERFITT, P.O. BOX 3188, WEST PALM BEACH, FL, 33402-3188, US

(73) Assignee: Fondazione Pierfranco e Luisa Mariani- ONLUS, Milano (IT)

(21) Appl. No.: 11/623,568

(22) Filed: Jan. 16, 2007

Related U.S. Application Data
(63) Continuation-in-part of application No. 11/324,056, filed on Dec. 30, 2005, now abandoned.

(51) Int. Cl. A61K 39/08

(52) U.S. Cl. 424/239.1

(57) ABSTRACT

A method for functionally identifying an epileptogenic focus in pre-surgical evaluation in affected subjects with intractable epilepsy is described, the method including the delivery of an effective dose of a botulinum neurotoxin (BoNT) to a presumptive epileptogenic focus in the disease-compromised central nervous system of a mammal, under conditions whereby the effective dose of the botulinum neurotoxin interacts with the soluble N-ethylmaleimide-sensitive factor-attachment receptor (SNARE) proteins, thus impairing neurotransmission.

### CROSS-REFERENCE TO RELATED APPLICATIONS

This application is a continuation in part of U.S. patent application Ser. No. 11/324,056 filed on Dec. 30, 2005, by Caleo et al.

### FIELD OF THE INVENTION

The present invention is concerned with a method for the mapping of the epileptogenic focus in the pre-surgical evaluation of patients suffering from a form of epilepsy resistant to pharmacological treatment. In particular, the invention relates to a method for the diagnostic determination of the site of origin of epileptic seizures, involving the delivery of a botulinum neurotoxin (BoNT).

### BACKGROUND OF THE INVENTION

Epilepsy is the most common serious neurological condition, with a prevalence of between 0.5 and 1% in developed countries and a lifetime cumulative incidence of 2-3%. In the United States, epileptic seizures affect about 1% of the population. (Hauser W A. Incidence of epilepsy and unprovoked seizures in Rochester, Minn., 1935 through to 1984. Epilepsia 1993; 34:353-68.). Seizures are a symptom of a disease affecting the central nervous system (CNS) circuits and which is characterized by an abnormal electrical discharge (excessive synaptic activity) of brain cells. In more detail, epileptic seizures are the manifestation of abnormal hypersynchronous discharges of central neurons. The clinical signs or symptoms of seizures depend upon the location of the epileptic discharge and its propagation along neural pathways. The causes of the disease can include brain damage before or at birth, brain trauma, tumors, strokes, infections, metabolic disorders, or genetic defects.

The conventional classification of epilepsies is based on the localization of the epileptogenic focus, that is, the point of origin of seizures. In generalized seizures, the electrical discharge begins simultaneously in both cerebral hemispheres, whereas in focal (or partial) seizures the discharge starts in one area of the brain from which it can propagate to other areas. Generalized seizures usually have a genetic cause and include generalized tonic-clonic (stiffening and jerking) or grand mal seizures. Partial seizures are subclassified according to the site of the focus (frontal, temporal, parietal and occipital lobe epilepsies) and are further divided according to aetiology. Idiopathic epilepsies are purely genetic and present primarily in childhood. Symptomatic epilepsies are symptomatic of a lesion, such as mesial temporal lobe sclerosis, cortical dysplasia or other malformations of cortical development (MCDs), tumour, vascular malformation, hemorrhage, infarct, infection and trauma. Cryptogenic epilepsies are those thought to be symptomatic but for which the precise underlying aetiology has not been determined. Partial epilepsy is the most common seizure disorder; the most frequently occurring seizure-type in the adult patient is a complex partial seizure of mesial temporal lobe origin (Dreifuss, F. E., 1987. Goals of surgery for epilepsy. In: Engel Jr., J. (Ed.), Surgical Treatment of the Epilepsies, first ed. Raven Press, New York, pp. 3149).

Conventional treatment consists of anticonvulsant medication. The selection of anticonvulsants is based on considerations over the seizure type and the specific epileptic syndrome of the individual. Carbamazepine represents the first line of therapy in the treatment of focal onset seizures although phenytoin, primidone, and phenobarbital have similar efficacies. Lamotrigine, topiramate, tiagabine, gabapentin, levetiracetam, oxcarbazepine, and zonisamide are considered for second- or third-line therapy. Non-pharmacological approaches also exist and include mainly ketogenic diet and vagal nerve stimulation (VNS).

Although for the majority of people with epilepsy the chances of successfully controlling the seizures is good, between 20 and 30% of the population will continue to suffer seizures despite undergoing treatment with one or more antiepileptic drugs (AEDs). Of the 20-30% of people who continue to have seizures despite drug treatment, the majority have a symptomatic or cryptogenic partial epilepsy. Seizures of focal origin that do not respond to AED treatment can be cured by surgical ablation of the affected brain regions (epilepsy surgery). It has been estimated that about 55-70% of temporal lobe and 30-50% of frontal lobe resections result in seizure remission after surgery (National Institutes of Health Consensus Conference, 1990. Surgery for epilepsy. JAMA 264, 729-733). The surgical outcome is distinctly less favorable in individuals with focal cortical dysplasia and other malformations of cortical development (Palmini, A., Andermann, F., Olivier, A., et al., 1991. Focal neuronal migrational disorders and intractable partial epilepsy: results of surgical treatment. Ann. Neurol. 30, 750-757). The most common surgical strategy in patients with intractable partial epilepsy involves a focal resection of the epileptogenic zone. Patients with medial temporal lobe epilepsy and lesional epileptic syndromes are the most suitable candidates for ablative surgical procedures. The most common surgical procedure involves resection of the epileptic brain tissue (i.e., focal corticectomy, resection of the anterior temporal lobe). The rationale behind the surgical treatment is the excision of the epileptogenic zone, i.e. the site of seizure onset and initial seizure propagation (Dreifuss, 1987; Engel Jr., J., Ojemain, G. A., 1993. The next step. In: Engel Jr., J. (Ed.), Surgical Treatment of the Epilepsies, second ed. Raven Press, New York, pp. 319-329). Excision of the epileptogenic zone is performed by standard neurosurgical procedures as well as by radiosurgery (Romanelli P., Anschel D. J., 2006. Lancet Neurol. 5, 613-620).

Currently, about one third of epilepsy surgery interventions are ineffective in curing the disease. An important reason for the unfavorable surgical outcome is the inherent difficulty in identifying the epileptogenic zone. This is particularly true for those types of partial seizures in which the anatomical location of the epileptogenic zone in these individuals involves extrahippocampal areas such as the neocortex. The most frequent site of seizure onset in patients with neocortical non-lesional partial epilepsy is the frontal lobe. In these patients, precise determination of the epileptogenic area may be difficult with the techniques available today. As a consequence of the inaccuracy of the pre-surgical evaluation, only a minority of patients with neocortical, extratemporal seizures are rendered seizure-free following surgical treatment (Cascino, G. D., Jack Jr., C. R., Parisi, J. E., et al., 1992. MRI in the pre-surgical evaluation of patients with frontal lobe epilepsy and children with temporal lobe epilepsy: pathological correlation and prognostic importance. Epilepsy Res. 11, 51-59).

Indeed, it is widely accepted that the effectiveness of epilepsy surgery is mostly based on the results of pre-surgical evaluation of those patients which—due to their refractory epileptic condition—are considered good candidates for surgery. The rationale for the pre-surgical evaluation is to identify the site of ictal onset and initial seizure propagation, i.e. the epileptogenic zone (Engel Jr., J., Ojemann, G. A., 1993). Pre-surgical procedures are currently run according to standard protocols, with the aim of accurately mapping the epileptogenic focus by a series of sequential investigations. The most important factor for the successful outcome of epilepsy surgery is appropriate patient selection. The first step is to take a detailed history and examination of the patient, with particular attention to the type and chronology of ictal clinical subjective and objective manifestations and the possible post-ictal deficits. This should allow the selection of patients who have or may have seizures arising from the temporal lobe and those who do not. Following this first evaluation, patients undergo a series of routine examinations, which can be outlined as follows (P Whiting, R Gupta, J Burch, R E Mujica Mota, K Wright, A Marson, U Weishmann, A Haycox, J Kleijnen and C Forbes, 2006. A systematic review of the effectiveness and cost-effectiveness of neuroimaging assessments used to visualise the seizure focus in people with refractory epilepsy being considered for surgery. Health Technology Assessment 10, 1-5; Cascino G. D. 2004. Surgical treatment for epilepsy. Epilepsy Research 60, 179-186):
1. standard electroencephalographic analysis (scalp EEG)
2. simultaneous video and EEG seizure recording (video-EEG, extracranial)
3. magnetic resonance imaging (MRI) of the head
4. neuropsychological/neuropsychiatric tests

Routine EEG recordings from the scalp allow to roughly identify the region of seizure onset. However, there are several limitations to the use of this technique in the identification of the anatomic source of epileptiform discharges in patients with intractable partial epilepsy. The electrical activity recorded by electrodes placed on the scalp or surface of the brain mostly reflects the summation of the electrical activity of a large number of neurons, mainly placed in the more superficial layers of the cortex. Quite large areas of the cortex—in the order of a few square centimetres—have to be activated synchronously to generate enough potential for changes to be registered at the electrodes placed on the scalp. Propagation of electrical activity along physiological pathways or through volume conduction in extracellular spaces may give a misleading impression as to the location of the source of the electrical activity. Moreover, spatial sampling in routine scalp EEG is incomplete, as significant amounts of cortex, particularly in basal and mesial areas of the hemispheres, are not covered by the standard electrode placement (Smith S. J. M., 2005. EEG in the diagnosis, classification, and management of patients with epilepsy. J. Neurol. Neurosurg. Psychiatry 76, 2-7). Simultaneous video and scalp-EEG seizure recording (ictal EEG) can greatly improve seizure focus determination, since it allows to correlate the electrographic and behavioural pattern of occurring seizures. For this examination, which is still non-invasive, patients are admitted to hospital for one week or more for continuous monitoring and usually have their medication reduced to increase the likelihood of seizures.

Neuroimaging technologies can provide information about structural abnormalities and the underlying aetiology of seizures, potentially allowing a precise determination of the epileptogenic focus. Routine magnetic resonance imaging (MRI) is particularly recommended for all patients with refractory focal epilepsy. In these patients, MRI can show features of mesial temporal sclerosis and also pick up lesions such as tumours, vascular malformations, developmental malformation as well as lesions resulting from trauma or infections.

Neuropsychological/neuropsychiatric evaluation include psychometric testing (memory and IQ) to find evidence of focal cognitive dysfunction, and neuropsychiatric tests (particularly in children) to determine any behavioural psychiatric abnormalities.

These routine investigations may be followed, in a certain number of cases, by more detailed analyses, to allow a more precise pre-surgical determination of the epileptogenic focus. These analyses include:

1. simultaneous video and intracranial EEG seizure recording

2. functional imaging studies (FDG-PET, interictal-ictal SPECT)

3. sodium amytal study

Invasive studies with stereotactically introduced intracerebral electrodes (stereo-EEG, simultaneous to video recording) are routinely performed in patients suffering from drug-resistant focal epilepsy, when it is considered necessary to resolve the borders of the epileptogenic zone to plan a surgical resection tailored to individual anatomical and electro-clinical features of the seizure-onset area (de Curtis M., Tassi L., Lo Russo G., Mai R., Cossu M., Francione S., 2005. Increased discharge threshold after an interictal spike in human focal epilepsy. Eur. J. Neurosci. 22, 2971-2976.; Whiting et al., 2006). Intracranial EEG recordings can be obtained via the implantation of i) single or multiple depth electrodes (inserted into selected brain areas via a small hole drilled into the skull), ii) foramen ovale electrodes (electrodes passed through the foramen ovale to lie under the temporal lobe), iii) subdural mats (grid of electrodes placed over an area of the cerebral cortex, following craniotomy) and iv) sphenoidal electrodes (thin, sterilised, disposable wires inserted into the soft tissues anterior to the temporo-mandibular joint using a needle). Following the recording of spontaneous seizures, intracerebral electrical stimulations can also be delivered, to provide a functional mapping of eloquent structures and/or to induce seizures, with the aim of better defining the epileptogenic zone.

Functional imaging studies can reveal the activation of selected brain areas before, during or after seizures, depending on metabolic activation. Positron emission tomography (PET) technology uses radiolabelled tracers, typically providing information about glucose metabolism using 18F-labelled fluorodeoxyglucose (FDG). Single photon emission computed tomography (SPECT) also uses a radiolabelled compound, which, depending on its properties, binds preferentially to certain areas of the brain. SPECT scans provide information about the uptake of the tracer. Most commonly, the epilepsy surgery work-up uses 99 mTc-labelled compounds [hexamethylpropylenamine oxime (HWPAO) or ethyl cysteinate dimer (ECD)] to map cerebral blood flow. Interictal scans (not during a seizure) often show an area of reduced uptake at the site of seizure onset. More reliable information about the site of seizure onset is provided by injecting the radiolabelled compound at the start of a seizure (ictal) or just after it (postictal). In this case, scans show an area of increased uptake at the site of seizure activity.

Sodium amytal study (also known as Wada test) is an invasive procedure to evaluate cognitive performance. In this test, a short-acting barbiturate (sodium amytal) is injected into one carotid artery to anaesthetise part of one hemisphere, and memory and language are then tested in the opposite hemisphere. The procedure is repeated for the other hemisphere.

Further studies can be performed in specialized centres to improve pre-surgical evaluation, if needed. These studies include PET receptor studies (with radioligands specific for brain receptor subtypes), MRI volumetry and subtraction ictal SPECT co-registered to MRI (SISCOM). Volumetric MRI involves stereological techniques applied to the analysis of MRI scans, allowing to estimate the volume of brain structures (most commonly the hippocampus, amygdala and temporal lobe). Differences in volume (usually a reduction) when compared with normative data suggest focal pathology that may be the site of the seizure onset. In SISCOM, the interictal SPECT images are subtracted from the ictal SPECT images and the resultant functional image is superimposed over the individual's MRI to combine the functional and structural data.

The Applicants' research activity has been focused for a long time on the evaluation of new diagnostic approaches based on neurotoxins, mainly those obtained from Clostridium botulinum.

Botulinum neurotoxins (BoNT), synthesized by various Clostridium botulinum strains are known to interfere with neural transmission. They are bacterial metallo-proteases acting on peripheral cholinergic terminals of the peripheral nervous system. They cause a long-lasting inhibition of the release of the neurotransmitter acetylcholine, which is responsible for transmission of stimuli. They act in the cytosol by cleaving core proteins of the neuroexocytosis apparatus named soluble N-ethylmaleimide-sensitive factor-attachment receptors (SNAREs).

There are seven neurotoxin serotypes overall, conventionally indicated by letters A to G. The botulinum neurotoxin is produced by Clostridia in the form of a single 150 kDa inactive polypeptide chain. Bacterial or tissue proteases then cleave the toxin transforming it into the active double chain form. In this active form, it consists of a heavy chain (H, Mᵣ 100 kDa) composed of two domains of 50 kDa each and a light chain (L, Mᵣ 50 kDa) bound by a disulphide bridge (part of the heavy chain being coiled around the light chain) and non-covalent bonds.

Cell intoxication by BoNTs takes place in four steps: first, the heavy chain of the toxin binds to the presynaptic membrane of neurons, where a polysialoganglioside and a protein are involved in the binding mechanism; the toxin then enters the cells from vesicles that are low in pH; it then translocates across the vesicular membrane; finally, the pH drop causes the disulphide bridge to be reduced thus causing the release of the light chain of the toxin into the cytoplasm, where enzymatic activity by its amino terminal blocks neuroexocytosis.

The light chains are metallo-proteases, more specifically zinc endopeptidases. It has been found that neurotoxins of serotype B, D, F and G cleave at different points a protein (VAMP/synaptobrevin) of the synaptic vesicles, whereas serotypes A and E cleave the synaptosomal-associated protein of 25 kDa (SNAP-25), and serotype C cleaves both SNAP-25 and syntaxin. In particular, SNAP-25 is of interest because it is a soluble protein which plays a key role in vesicle membrane fusion events with the plasma membrane and displays a presynaptic pattern of expression. Proteolytic cleavage of either VAMP, SNAP-25 or syntaxin by the toxin prevents the formation of the trimeric SNARE complex, which is required for synaptic vesicle release and neuroexocytosis.

The discovery of the effect of these toxins on the neuromuscular junctions has promoted their therapeutic and medical use in pathologies involving hyperactivity of the neuromuscular junctions, which benefit from the partial inhibition of synaptic activity upon topical injection of very small quantities of the toxin. Botulinum toxin serotype A (Botox®) was approved by the US Food and Drug Administration in December 1989 for three disorders (strabismus, blepharospasm, and hemifacial spasm) and is currently used for a variety of treatments related to muscular pathologies, including spasticity, tremors, migraines and even cosmetic treatment. The effects are reversible and last for 2 to 6 or 8 to 12 months depending on the muscles involved. Serotype C has a comparable length of action. Serotypes B, D, E, and F, instead, have a shorter effect.

U.S. Pat. No. 6,306,403 by Donovan describes a method for treating a movement disorder, epilepsy included, by intracranial administration to a human patient of a therapeutically effective amount of a neurotoxin, such as a botulinum toxin. No mention, however, is made of the use of such a toxin as a pre-surgical tool for the localization of the epileptogenic focus.

The Applicants explored the possibility of exploiting the inhibitory effects of the Botulinum toxins on neurotransmission for the pre-surgical evaluation of the epileptogenic focus in epileptic patients, in particular those resistant to conventional antiepileptic drugs.

The Applicants found that the Botulinum neurotoxins, in particular the serotype E, actually showed a potential for such a use. As a matter of fact, experiments on Long-Evans hooded rats have shown that injecting botulinum neurotoxin E (BoNT/E) into the hippocampus inhibits glutamate release and blocks spike activity of pyramidal neurons. It reduces both focal and generalized kainic acid (KA)-induced seizures BoNT/E application also prevents neuronal loss and long-term cognitive deficits associated with kainic acid seizures. BoNT/E delivery is thus anti-ictal in experimental models of epilepsy.

Based on these findings, the Applicants propose that the anticonvulsant properties of BoNTs may be exploited for the pre-surgical identification of the epileptogenic focus in patients with intractable epilepsy.

### SUMMARY OF THE INVENTION

The invention provides a diagnostic method for the evaluation of candidate patients for epilepsy surgery. Specifically, the method is used to functionally validate the location of an epileptogenic focus that is previously achieved with both non-invasive and invasive techniques. Surgical success rate in drug-resistant patients is directly related to the ability to precisely locate the area of seizure onset. In this respect, the present method allows to determine whether the candidate patient has a single resectable region of the brain that is responsible for seizure origin. This is of invaluable help in the selection of patients for surgery since the area of resection can be accurately selected and the rate of successful surgeries can be dramatically increased.

The invention comprises the delivery of an effective dose of a botulinum neurotoxin to a presumptive epileptogenic focus in a region of the disease-compromised central nervous system (CNS) of a mammal, under conditions whereby said effective dose of BoNT interacts with the soluble N-ethylmaleimide-sensitive factor-attachment receptor (SNARE) proteins, thus impairing neurotransmission.

Accordingly, in one aspect, the present invention provides a method for mapping the epileptogenic foci in pre-surgical evaluation in patients with epilepsy. The methods of the invention are suitable for application in populations which include patients suffering from epileptic seizures of various location and aetiologies.

In one aspect, the disclosed methods include delivering an effective dose of BoNT to a presumptive epileptogenic focus in the CNS of the afflicted subject.

According to an aspect, the method comprises delivery of an effective dose of BoNT to a presumptive epileptogenic focus located in one of the following regions of the CNS of the afflicted subject: the cerebral cortex, the frontal lobe, the parietal lobe, the temporal lobe, the occipital lobe, the striatum, the hippocampus, the amygdala, the thalamus, the hypothalamus, the mesencephalon, the cerebellum, the brainstem, the pons, the medulla, the spinal cord.

In illustrative aspects, the administration is accomplished by direct injection into the hippocampus or cerebral cortex of an affected brain of a composition comprising an effective dose of the botulinum neurotoxin.

According to a preferred aspect, following identification of the seizure origin using non-invasive methodologies, the method of the invention comprises the steps of:
a) Determining a location of a presumptive epileptogenic focus by means of one or more techniques selected from electroencephalographic techniques, magnetic resonance imaging and tomographic techniques;
b) Injecting a botulinum neurotoxin (BoNT) solution to the presumptive epileptogenic focus in a subject through an intracerebral cannula, the cannula being fixed to an electrode that allows low-frequency bipolar stimulation;
c) Checking the spread of the neurotoxin and the coverage of the presumptive area of seizure onset;
d) Routine checking for occurrence of spontaneous seizures; and
e) Defining the limits of the surgical resection.

Preferably, the cannula is a magnetic resonance-compatible cannula and its intraparenchymal trajectory is planned on stereo-angiographic and three-dimensional magnetic resonance images.

Preferably, the electrodes are multi-lead intracerebral electrodes having a diameter of 0.8 mm and consisting of 5 to 15 leads, each 2 mm long with an interelectrode distance of 1.5 mm (Francione et al., J Neurol Neurosurg Psychiatry 74: 1493, 2003).

Preferably, the neurotoxin is injected in an amount that varies between 0.002×10⁻⁶ and 0.2×10⁻⁶ mg/kg of the affected subject, preferably between 0.01×10⁻⁶ and 0.15×10⁻⁶ mg/kg, more preferably 0.1×10⁻⁶ mg/kg.

Preferably, the neurotoxin solution is injected in a volume that varies between 0.1 and 10 μl, preferably between 0.1 and 1 μl, more preferably, in a volume of 0.5 μl.

Preferably, assessment of the spread of the neurotoxin and of the coverage of the presumptive area of seizure onset is checked via functional neuroimaging over a period of time of 1 to 15 days, preferably 3 to 10 days post injection.

Preferably, routine checking for occurrence of spontaneous seizures is carried out over a period of time of 1 to 15 days, preferably 3 to 10 days post injection.

According to another preferred aspect, whenever the seizure origin cannot be identified using non-invasive methodologies, the method of the invention comprises the steps of:

a) implanting a plurality of intracerebral electrodes that allow low-frequency bipolar stimulation in a subject, each one of the electrodes being fixed to an intracerebral cannula;

b) chronic stereo EEG monitoring and recording of spontaneous or induced seizures to define the presumptive epileptogenic zone;

c) intracerebrally injecting, via the cannulas, a solution of a botulinum neurotoxin (BoNT) near the electrode tips located in the area of presumptive seizure onset;

d) post injection chronic stereo EEG monitoring, combined with functional neuroimaging to assess the spread of the neurotoxin effects;

e) confirming or rejecting presumptive localization of the epileptogenic focus on the basis of the ability of the neurotoxin to suppress seizures.

Preferably, the suitable intracerebral electrodes are selected from the group comprising: depth electrodes, wherein wires are inserted into the brain via a small hole drilled into the skull; foramen ovale electrodes, wherein wires are passed through the foramen ovale to lie under the temporal lobe; subdural mats, wherein grids of electrodes are placed over an area of cerebral cortex following a craniotomy; and sphenoidal electrodes, wherein thin wires are inserted into the soft tissues anterior to the temporo-mandibular joint using a needle.

Preferably, the neurotoxin is injected in an amount that varies between 0.002×10⁻⁶ and 0.2×10⁻⁶ mg/kg of the affected subject, preferably between 0.01×10⁻⁶ and 0.15×10⁻⁶ mg/kg, more preferably 0.1×10⁻⁶ mg/kg.

Preferably, the neurotoxin solution is injected in a volume that varies between 0.1 and 10 μl, preferably between 0.1 and 1 μl, more preferably, in a volume of 0.5 μl.

Preferably, the chronic stereo EEG monitoring and recording of step b) is carried out over a period of time of 1 to 15 days, more preferably between 3 and 10 days.

Preferably, the chronic stereo EEG monitoring of step d) is carried out over a period of time of 1 to 15 days following neurotoxin administration; more preferably between 3 and 10 days following neurotoxin administration.

Preferably, seizures are induced by electrical stimulation.

Preferably, step d) consists of identifying the blocked area by functional neuroimaging.

The method of delivering an effective dose of BoNT to the synaptic terminals in an affected brain, comprises exposing a synaptic terminal membrane of the neuron of the affected brain to an effective dose of BoNTs and allowing the BoNTs to bind to the membrane and enter the cell via membrane vesicles. The toxin is then translocated across the vesicle membrane by mediation of the intermediate domain of the protein, and the light chain of the protein is then released by the reduction of the disulphide bridge, and blocks neuroexocytosis.

In the method, BoNTs interfere with neurotransmitter release in a very specific manner through its interaction with the soluble N-ethylmaleimide-sensitive factor-attachment receptor (SNARE) proteins thus impairing neurotransmission. Furthermore, BoNTs potently inhibit the spiking activity of central nervous system (CNS) neurons.

The effective dose of BoNT is comprised between 0.002×10⁻⁶ and 0.2×10⁻⁶ mg/kg of the affected subject.

It is to be understood that both the foregoing general description and the following detailed description are exemplary and explanatory only and are not restrictive of the invention as claimed.

### BRIEF DESCRIPTION OF THE DRAWINGS

FIG. 1 illustrates the characterization of BoNT/E effects in the hippocampus. (A) Representative immunoblotting for cleaved SNAP-25 from the BoNT/E-injected and contralateral, not injected hippocampus of a P36 rat. β-tub, β-tubulin (internal standard); (B) Forebrain coronal section showing the hippocampus of a P36 rat, one day after unilateral BoNT/E injection; (C) Effects of BoNT/E on Ca²⁺-dependent, 35 mM K⁺-evoked glutamate overflow from hippocampal synaptosomes prepared 3 days after toxin injection. Data are expressed as mean±S.E. (n=7 animals per group; **, t-test, p<0.01); (D) Representative recordings of spontaneous spike activity from CA1 in vehicle- and BoNT/E-injected animals; (E) Immunoblotting for cleaved SNAP-25 on protein extracts from hippocampi of BoNT/E-treated rats at different times after BoNT/E injection. D, dorsal; V, ventral hippocampus; (F) Immunoblotting for the intact SNAP-25 (S25) from hippocampi of BoNT/E-treated and age-matched control rats at different times after BoNT/E injection;

FIG. 2 illustrates the anti-ictal effect of BoNT/E on EEG (electroencephalographic) seizures induced by intrahippocampal KA. (A) Three-dimensional drawing of the rat brain illustrating the location of BoNT/E injection sites and the placement of KA injection cannula and recording electrodes. Position of the cortical electrode is indicated by an asterisk; (B) Nissl-stained coronal section through the hippocampus indicating the sites of BoNT/E injection and the position of the KA infusion cannula glued to the recording electrode (X); (C-D) EEG tracings depicting seizure activity induced by unilateral injection of 40 ng KA into the dorsal hippocampus of rats that received 2 days before the convulsant either vehicle (C) or BoNT/E (D). Traces on the left represent baseline recordings in the parietal cortex (CTX), right hippocampus (RHP) and left hippocampus (LHP). Traces on the right depict typical seizure episodes. Time elapsed from the KA injection is indicated; (E-G) Quantification of EEG seizures in control (black circles) and BoNT/E-injected (open circles) animals. Each point indicates one animal and horizontal bars represent mean values (±S.E.);

FIG. 3 shows a comparison of the anti-ictal effects of phenytoin (PHT) and intrahippocampal BoNT/E. *, p<0.05; **, p<0.01; one way ANOVA followed by Tukey test;

FIG. 4 illustrates the results of the investigation on the effect of BoNT/E on seizures induced by systemic KA. (A) Progression of behavioral changes after systemic KA administration. Data are mean seizure scores±S.E; (B) Scatter plot reporting the maximum seizure score assigned to each experimental animal during a 4 hr observation period following KA administration. **, p<0.01; one way ANOVA followed by Dunn's test.

FIG. 5 shows the results of the investigation on the effect of BoNT/E on spatial learning deficits induced by KA. (A) Acquisition of place learning in the Morris water maze (MWM) for rats injected with BoNT/E into the hippocampus at P35 and tested 3-7 days later (BoNT/E, n=8; black circles); the performance of vehicle-treated rats (control, n=8; open circles) is also shown. Time required to find the submerged platform (escape latency) is indicated in seconds and plotted as mean values (±S.E.) across days of testing; (B) Acquisition of place learning in the MWM for rats injected with BoNT/E into the hippocampus at P35 and tested five weeks later (BoNT/E, n=5; black circles); the performance of naive age-matched rats (control, n=10; open circles) is also shown; (C) Acquisition of place learning in the MWM for rats injected with BoNT/E at P35 and injected with KA at P36 (BoNT/E+KA, n<13; open triangles); the performance of vehicle-injected rats treated with KA is also shown.

FIG. 6 shows the results of the investigation on the effect of BoNT/E on neuronal loss induced by KA. (A) Representative coronal sections through the dorsal hippocampus of P77 rats that received vehicle (top) or BoNT/E (bottom) at P35 and KA at P36. The injected side is indicated by an asterisk. (B) Representative pictures showing details of CA1 and CA3 regions of the hippocampus from vehicle- and BoNT/E-injected rats. (C) Quantification of histological lesions in the dorsal hippocampus of control (black circles) and BoNT/E-injected (open circles) rats treated with KA; **, p<0.01, Mann-Whitney rank sum test.

### DETAILED DESCRIPTION

A definition of some of the terms used throughout the present application will now be provided in the interest of aiding the comprehension of its content.

The expression “effective amount or dose” refers to the amount of a compound that results in an attainment of a desired biological outcome.

In one aspect, the present invention provides a method for the identification of the area of seizure onset in a mammal. The method of the invention is suitable for application in populations that include patients affected by epileptic seizures, in particular focal-onset seizures.

In some aspects, the method comprises delivery of an effective dose of BoNT to a presumptive epileptogenic focus in the CNS of the affected subject.

According to an aspect, the method comprises delivery of an effective dose of BoNT to a presumptive epileptogenic focus located in one of the following regions of the CNS of the afflicted subject: the cerebral cortex, the frontal lobe, the parietal lobe, the temporal lobe, the occipital lobe, the striatum, the hippocampus, the amygdala, the thalamus, the hypothalamus, the mesencephalon, the cerebellum, the brainstem, the pons, the medulla, the spinal cord.

In a preferred aspect of the invention, the method comprises delivery of an effective dose of BoNT to the seizure focus in the brain of the affected subject.

In a particularly preferred aspect of the invention, the method comprises delivery of an effective dose of BoNT to the synaptic terminals of hippocampal and/or cortical neurons of an affected brain.

To deliver the effective dose specifically to a particular region of the central nervous system, especially to a particular region of the brain, it may be administered by stereotaxic microinjection. The affected subjects have a stereotaxic frame base fixed to the skull and the brain is imaged using high resolution MRI. The images thus obtained are then transferred to a computer running stereotaxic software and a series of images allow the determination of the target site of toxin injection. Holes are drilled above the entry site and the stereotaxic apparatus placed with the needle implanted at a suitable depth. The toxin is then injected.

In a particularly preferred aspect of the invention, in particular in those cases where non-invasive techniques are ineffective or contradictory in revealing the exact seizure focus, affected subjects that are eligible for epilepsy surgery are implanted with intracranial depth electrodes, each of which is glued to an infusion cannula for intracerebral delivery of BoNT. Stimulation is performed to identify the epileptogenic zone and functionally map the explored region. After a period of EEG monitoring, BoNT is injected at selected locations and EEG monitoring is continued to check whether spontaneous seizures are stopped.

The total volume of material to be delivered, and the concentration of the solution will be determined by those skilled in the art based upon known aspects of epilepsy treatment. In experimental rats, the total volume administered was 1.5 μl of BoNT/E (50 nM). For other mammals, including humans, volumes and delivery rates should be scaled appropriately with the volumes being generally comprised between 0.1 and 10 μl. The effective dose of BoNT is comprised 0.002×10⁻⁶ and 0.2×10⁻⁶ mg/kg of the affected subject. Administration may consist of a single injection per target site of or may be repeated along the injection tract if necessary. Multiple injection sites may be used.

BoNT is derived from Clostridium botulinum according to standard practices known in the art and is purified according to Schiavo and Montecucco (Schiavo G., Montecucco C. (1995) Methods Enzymol 248:643-652).

BoNT can be used in the form of a frozen solution of purified BoNT (Mᵣ 150 kDa) at a concentration comprised between 1 and 300 nM in a phosphate buffered saline (PBS) solution.

The solution can also contain between 0.5 and 4% (w/v) serum albumin.

The solution is usually stored at −80° C. and is thawed on ice and stored refrigerated until injected. The surgical procedure is carried out at room temperature (18-25° C.).

The following examples provide illustrative aspects of the invention. One of ordinary skill in the art will recognize the numerous modifications and variations that may be performed without altering the spirit or scope of the present invention. Such modifications and variations are encompassed within the scope of the invention. The examples do not in any way limit the invention.

### EXAMPLES

In Examples 1-5, we report the results of investigations performed in Long-Evans hooded rats. Animals were housed in a 12 hr light/dark cycle with food and water available ad libitum. All experimental procedures were in conformity to the European Communities Council Directive no 86/609/EEC. In Examples 6-7, we exploit the animal data and report a detailed procedure for the functional mapping of an epileptogenic focus in humans.

### Example 1

### Characterization of BoNT/E Actions in the Hippocampus

BoNT/E Injections

BoNT/E was obtained by WAKO (Japan), trypsin activated, purified and tested as described in Schiavo and Montecucco (Schiavo G., Montecucco C. (1995) Methods Enzymol 248:643-652). Its potency was evaluated with the mice phrenic nerve-hemidiaphragm test. Two unilateral stereotaxic infusions of 1.5 μl of BoNT/E (50 nM) or vehicle (2% rat serum albumin in PBS) were made into the dorsal hippocampus under avertin anaesthesia at postnatal day (P) 35. P35 rats were chosen since they display a maximal sensitivity to KA-induced seizures (Ben-Ari Y (1985) Neuroscience 14:375-403; Stafstrom et al. (1993) Epilepsia 34:420-432). Coordinates in mm from bregma were (nose bar −2.5): for CA1, AP −2.4, L+1.8, H 2.1 below dura; for CA3, AP −2.4, L+3.3, H 3 below dura.

Detection of Cleaved SNAP-25

Cleaved SNAP-25 was detected by immunostaining and immunoblotting using a peptide-affinity purified polyclonal antibody raised against the BoNT/E truncated C-terminal peptide of SNAP-25 (CDMGNEIDTQNRQIDR). This antibody recognizes specifically cleaved SNAP-25 but not the whole protein. For immunohistochemistry, rats received hippocampal injections of BoNT/E (n=10) or vehicle (n=5) and were transcardially perfused with 4% paraformaldehyde 1-4 days later (Caleo et al. (2003) J Neurosci 23:287-296). Brain coronal sections (40 μm thick, cut on a freezing microtome) were blocked with 10% normal goat serum in PBS and then incubated overnight with the anti-BoNT/E-cleaved SNAP-25 antibody diluted 1:300 in a PBS solution containing 1% serum and 0.3% Triton X-100. On the following day, sections were reacted with a biotinylated secondary antibody (Vector Laboratories, Burlingame, Calif.) followed by avidin-biotin-peroxidase complex (ABC kit, Vector Laboratories) and diaminobenzidine (DAB) reaction. For immunoblotting, rats (n=13) received hippocampal injections of BoNT/E and dorsal and ventral hippocampi (ipsilateral and contralateral to the injected side) were dissected after 1, 14, 21 and 35 days. Protein extracts (10 μg) were separated by electrophoresis and blotted, and filters were incubated with anti-BoNT/E-cleaved SNAP-25 (1:50 dilution) or anti-SNAP-25 (1:1,000 dilution; Synaptic Systems, Germany) polyclonal antibodies, reacted with HRP-conjugated goat anti-rabbit secondary antibody (Bio-Rad) and developed by ECL (Amersham, UK). Filters were stripped and re-probed with anti-β-tubulin monoclonal antibody (1:500 dilution; Sigma, St Louis, Mo.), which served as an internal standard for protein quantification.

Glutamate Release

Glutamate release measurements were performed on superfused hippocampal synaptosomes from vehicle- and BoNT/E-treated rats (n=7 per group), 1 day after intrahippocampal injection of 40 ng kainic acid (KA, see Example 2 below for details). Preparation of synaptosomes was as described by Gobbi et al. (2002) J Neurochem 82:1435-1443, and release of glutamate was assessed according to Di Stasi et al. (2002) J Neurochem 82:420-429. Synaptosomes were depolarised by a 90-sec pulse of 35 mM KCl. Glutamate overflow was measured by a Waters Alliance HPLC analysis system. The analytical method involved automatic precolumn derivatization with o-phthalaldehyde followed by separation on C18 reverse phase chromatography column and fluorimetric detection (Di Stasi et al. (2002) J Neurochem 82:420-429).

Recordings of Spike Activity

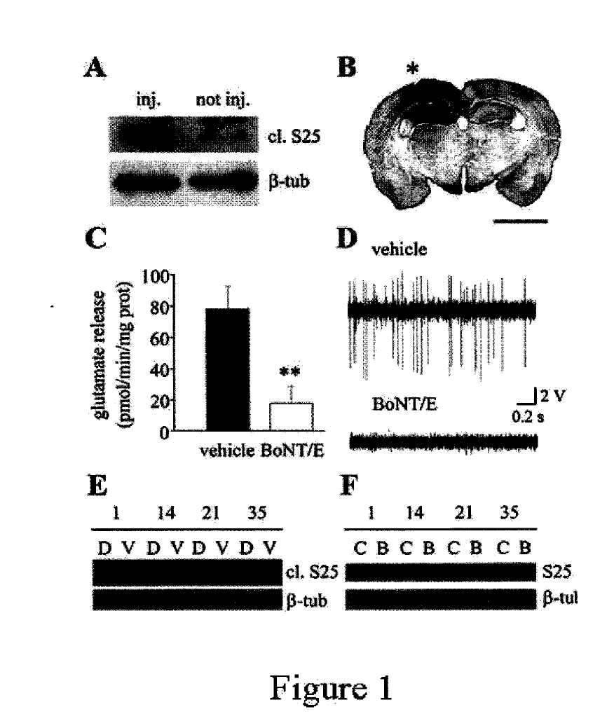

Rats received unilateral injections of BoNT/E (n=6) or vehicle (n=3). Recordings of spike activity were performed in the injected hippocampus 1-2 days after BoNT/E or vehicle injection as described by Caleo et al. (2003) J Neurosci 23:287-296. Animals were anesthetized with urethane (Sigma; 20% solution in saline; 0.7 ml/100 g body weight, i.p.) and placed in a stereotaxic frame. Body temperature was continuously monitored and maintained at 37° C. by a thermostat-controlled electric blanket. After exposure of the cerebral surface, a micropipette (tip resistance=2 MΩ) filled with 3M NaCl was inserted into the brain to reach the dorsal hippocampus. Two to five penetrations per hemisphere were made to map spike activity in CA1 and CA3 sectors (see FIG. 1D). Location of the recording sites was determined using histological controls (Caleo et al. (2003) J Neurosci 23:287-296). Signals were amplified 25,000 fold, band-pass filtered (500-5,000 Hz) and conveyed to a computer for storage and analysis.

Results:

Western blot for cleaved SNAP-25, performed 1 day after BoNT/E injection, demonstrated that SNAP-25 is efficiently proteolysed in vivo by BoNT/E (FIG. 1A). This effect was mainly restricted to the injected side and cleaved SNAP-25 was almost undetectable in the contralateral hippocampus (FIG. 1A). Immunostaining for cleaved SNAP-25 confirmed the regional specificity of the BoNT/E effect (FIG. 1B). Anteroposterior spread of staining around the injection site was of about 3 mm.

On the basis of the ex vivo experiments on superfused hippocampal synaptosomes, it was found that the Ca²⁺-dependent fraction of potassium-induced glutamate release was markedly reduced by BoNT/E administration (n=7 animals per group; Student's t-test, p<0.01; FIG. 1C).

In vivo recordings of spike activity from hippocampal pyramidal demonstrated that spontaneous discharges of spikes of high amplitude could be observed in the CA1 and CA3 sectors of naïve rats. These discharges were potently inhibited by BoNT/E treatment, while injection of vehicle solution had no effect (FIG. 1D). The reduction in activity was specific to the BoNT/E-treated hippocampus and no effects were found in the contralateral, uninjected side. Thus, BoNT/E impairs excitatory transmission in the hippocampus and the net electrophysiological effect is a silencing of spontaneous spike activity of pyramidal neurons.

Cleaved SNAP-25 was detected in both the dorsal and ventral hippocampus 1 day after BoNT/E injection. The band was slightly reduced at 14 days, persisted up to 21 days and was no longer detectable at 35 days (FIG. 1E). In keeping with the expression profile of cleaved SNAP-25, intact SNAP-25 was absent 1 day after BoNT/E, began to reappear at 14 days and was completely replenished by 35 days (FIG. 1F). Thus, BoNT/E effects persist for at least 3 weeks in the injected hippocampus. Importantly, BoNT/E-injected animals never showed any sign of systemic intoxication (i.e., muscular paralysis) during this time window.

### Example 2

### EEG (Electroencephalographic) Analysis after Intrahippocampal KA

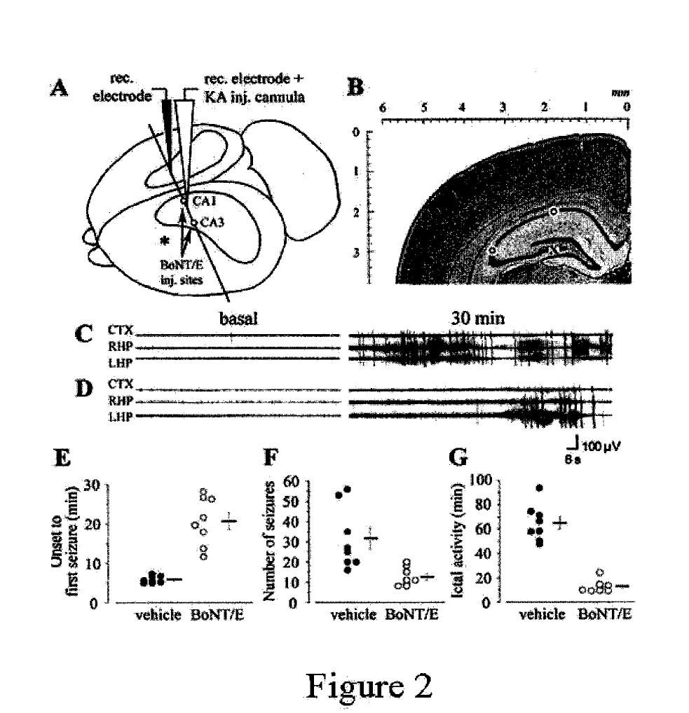

Rats were unilaterally infused into the left dorsal hippocampus with BoNT/E (n=8) or vehicle (n=8) as described above under avertin anaesthesia. At the end of the infusion procedure, one screw electrode was placed over the parietal cortex ipsilateral to the injected hippocampus together with a ground lead over the nasal sinus. Two depth bipolar electrodes made of insulated nichrome wire (60 μm) were implanted bilaterally into the dorsal hippocampus (nose bar −2.5; mm from bregma, AP, −2.4; L+1.8; H 3.0 below dura) and a guide cannula was glued to the left side depth electrode and positioned on top of dura for the intrahippocampal injection of KA. Surface and depth electrodes were connected to a multipin socket and secured to the skull together with the injection cannula by acrylic dental cement. Two days after surgery, freely-moving rats injected with BoNT/E or its vehicle received a unilateral injection of 40 ng KA into the left hippocampus using a needle protruding of 3 mm below the guide cannula (Vezzani et al. (1999) J Neurosci 19:5054-5065). The location of BoNT/E injection sites, KA infusion cannula and recording electrodes is schematically shown in FIG. 2A-B.

To compare the effect of BoNT/E with that of a conventional antiepileptic drug, one group of rats (not previously treated with BoNT/E) received phenytoin (PHT; 50 mg/kg, i.p.; n=7 rats) dissolved in propylene glycol, or vehicle (n=7), 60 min before hippocampal delivery of 40 ng KA.

EEG recordings on freely-moving animals were performed using a four-channel EEG polygraph, by an investigator who was unaware of the treatment of the animals. An initial 15-30 min recording was made to establish basal activity, then EEG recordings were made continuously up to 4 hours after KA administration. The KA dose (40 ng) was previously shown to induce EEG seizures recurring for about 180 min in 100% of the rats without mortality (Vezzani et al. (1999) J Neurosci 19:5054-5065). The EEG analysis was based on visual inspection of tracings to detect and quantify ictal activity. EEG seizures (ictal episodes) were defined by the occurrence of discrete episodes consisting of the simultaneous occurrence of at least two of the following alterations in cortical and hippocampal leads of recording: high frequency and/or multispike complexes and/or high-voltage synchronized spike or wave activity. The quantitative parameters chosen to quantify seizure activity were the latency to the first EEG seizure (onset), the total number of seizures occurring during the whole period of recording, and the total time spent in seizures which was reckoned by adding together the duration of all ictal episodes (Vezzani et al. (1999) J Neurosci 19:5054-5065). During EEG seizures the rats had the typical “frozen” appearance and apparently lost their reaction to external stimuli. “Wet dog shakes” were often observed at the end of seizure episodes. These behavioral sequelae were not quantified in this study. Statistical analysis was performed by Student's t-test and by two-way ANOVA followed by post-hoc Tukey test.

Results:

Rats were given unilateral injections of BoNT/E or vehicle into the hippocampus and seizures were induced two days later by focal KA application to the treated hippocampus. The location of BoNT/E injection sites, KA infusion cannula and recording electrodes is schematically shown in FIG. 2A-B.

EEG seizures occurred simultaneously in all leads of recordings and recurred as discrete episodes for about 180 min from their onset. Representative EEG tracings from vehicle or BoNT/E injected rats are shown in FIG. 2C-D. Quantification of seizure activity demonstrated that the onset time to seizures was delayed by 4-fold in BoNT/E-treated animals (Student's t-test, p<0.001; FIG. 2E). BoNT/E-injected rats showed a 2.6-fold reduction in the number of EEG seizures (Student's t-test, p=0.005; FIG. 2F), and a 5-fold decrease in the total time spent in seizure activity (Student's t-test, p<0.001; FIG. 2G). The duration of individual ictal episodes was also reduced by the toxin (min±S.E., vehicle, 2. 4±0.5; BoNT/E, 1.06±0.1, Student's t-test, p<0.01).

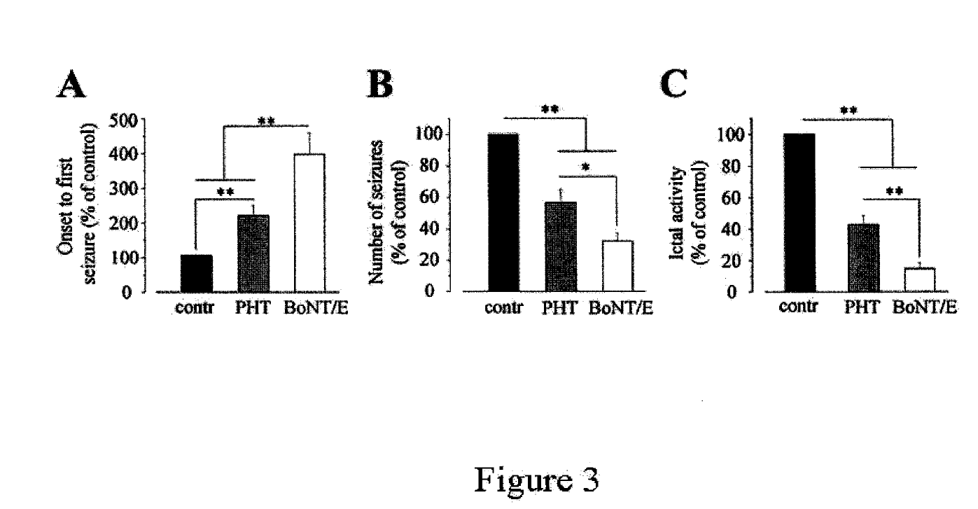

The anti-ictal effects of BoNT/E were compared with those of phenytoin (PHT), a standard anticonvulsant drug. PHT (50 mg/kg, i.p.; n=7 rats) was administered 60 min before intrahippocampal KA. This dose of PHT was chosen based on previous studies (Ebert et al. (2000) Neuropharmacology 39: 1893-903; N'Couemo P and Faingold CL (2000) Brain Res 859: 311-317) and was the highest dose that did not cause toxic effects in the animals (i.e., ataxia or sedation). PHT induced an average 2-fold delay in the onset of EEG seizures and a 2.5-fold reduction in the time spent in seizure activity (Student's t-test, p<0.01). PHT also reduced by 50% the number of EEG seizures (p<0.01; FIG. 3A-C). Thus, BoNT/E was significantly more effective than PHT in reducing KA-induced seizures (FIG. 3A-C; one way ANOVA, p<0.01, post hoc Tukey test, p<0.01 for onset and time spent in seizures; one way ANOVA, p<0.01, post hoc Tukey test, p<0.05 for number of seizures).

### Example 3

### Behavioral Analysis after Systemic KA

Behavioral, but not EEG analysis of seizures, was carried out in rats systemically injected with KA.

Thirty rats received hippocampal injections of BoNT/E at P35. Control animals of the same age (n=39) were injected with vehicle. One day after the injections, animals received a convulsive dose (8 mg/kg, i.p) of KA (Ocean Produce International, Shelburne, NS, Canada). Naïve animals which only received KA at P36 (n=20) were also used.

Rats were observed by an investigator unaware of the treatment. For each animal, behavior was scored every 5 minutes for a period of 4 hours after KA administration, according to a previously defined seizure rating scale (Schauwecker, P E and Steward, O (1997) Proc Natl Acad Sci USA 94:4103-4108; Bozzi et al. (2000) J Neurosci 20:8643-8649): stage 0: normal behavior; stage 1: immobility; stage 2: stereotypes; stage 3: wet dog shakes, head bobbing; stage 4: sporadic clonus of forelimbs with rearing and falling; stage 5: generalized clonus with continuous rearing and falling (status epilepticus); stage 6: death. Statistical analysis was performed by two-way ANOVA followed by post-hoc Tukey test.

Results:

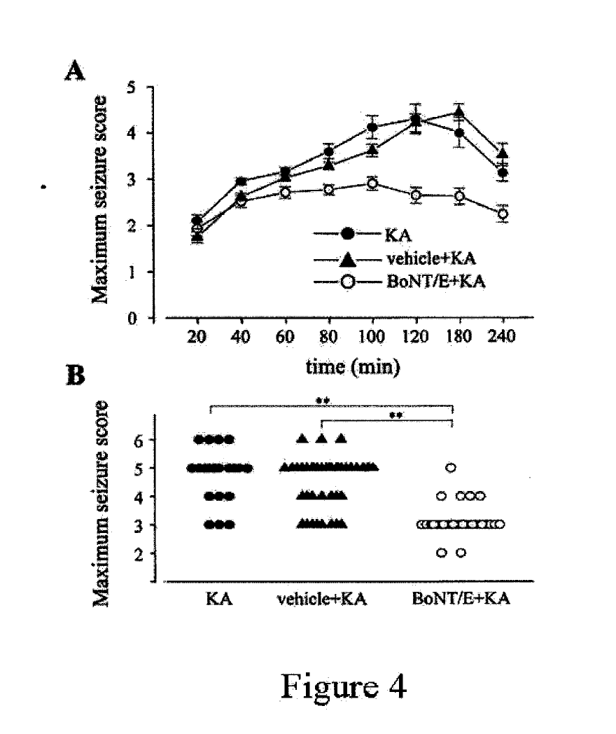

KA treatment had a similar convulsant effect in both naïve and vehicle-injected animals (two-way ANOVA, p>0.05; FIG. 4A). These rats showed initial immobility and staring followed by “wet dog shakes”, culminating in generalized clonic motor seizures with rearing and falling. Progression of clinical signs was dramatically different in BoNT/E-injected animals (FIG. 4A). Indeed, the trajectory in behavior score of BoNT/E-treated rats was dramatically different from that of control rats starting from 80 min following KA administration (two-way ANOVA, p<0.001; post hoc Tukey test, BoNT/E vs. vehicle and uninjected rats, p<0.01). In FIG. 4B, the results of the behavioral analysis are summarized as the maximum seizure rating scale value assigned to each animal during the 4 hours of observation following KA administration. KA triggered typical limbic motor convulsions in 17 out of 20 (85%) naïve rats and 31 out of 39 (79%) vehicle-injected rats. In contrast, the vast majority of the BoNT/E-injected rats showed only pre-convulsive behaviors, and only 5 out of 30 (16%) experienced limbic seizures upon KA administration. Analysis of variance demonstrates that the anticonvulsant effect of BoNT/E is highly significant (one way ANOVA, p<0.001; post hoc Dunn's test, p<0.01), while naïve and vehicle-injected rats do not show significant differences (p>0.05). Lethal toxicity induced by KA was also abolished by BoNT/E injection (FIG. 4B).

### Example 4

### Evaluation of Cognitive Performance in the Morris Water Maze

To evaluate the cognitive performance of rats during the time window of action of BoNT/E, 8 animals that received BoNT/E at P35 were tested in the Morris water maze beginning from 3 to 7 days after treatment. These animals were compared to vehicle-injected animals of the same age (n=8). A second group of rats (n=5) injected with BoNT/E at P35 were allowed to recover for 5 weeks before the spatial learning test. Their performance was compared to that of age-matched rats (n=10) which did not receive any treatment.

Cognitive performance of BoNT/E-injected and control rats treated with KA were also evaluated. Behavioral tests were begun on P70 (5 weeks after treatment) in both BoNT/E treated (n=13) and control (n=26; n=19 vehicle-injected and n=7 naïve) rats that received KA at P36.

Experiments were performed according to Mikati et al. (2001) Epilepsy Res 43:97-101, and Cilio et al. (2001) Neuropharmacology 40:139-147. Briefly, a circular tank (200 cm diameter) was filled with opaque water (22±1° C.), and a wooden platform (10×10 cm) was positioned in the center of one quadrant of the pool 2.5 cm below the water surface. On day 1 of testing, rats were placed in the pool for 60 s without the platform present, to become familiar with the training environment. Rats were trained for four days (five trials a day) to locate and escape onto the submerged platform. The latency from immersion into the pool to escape onto the platform was recorded for each trial. On mounting the platform, rats were given a 30 s rest period. Rats which did not find the platform in 120 s were placed on the platform for 30 s. Rats experiencing a spontaneous seizure during testing were allowed to recover for 60 min before resumption of test. All experiments were conducted in a blinded fashion. Statistical analysis was performed by two-way ANOVA followed by post-hoc Tukey test.

Results:

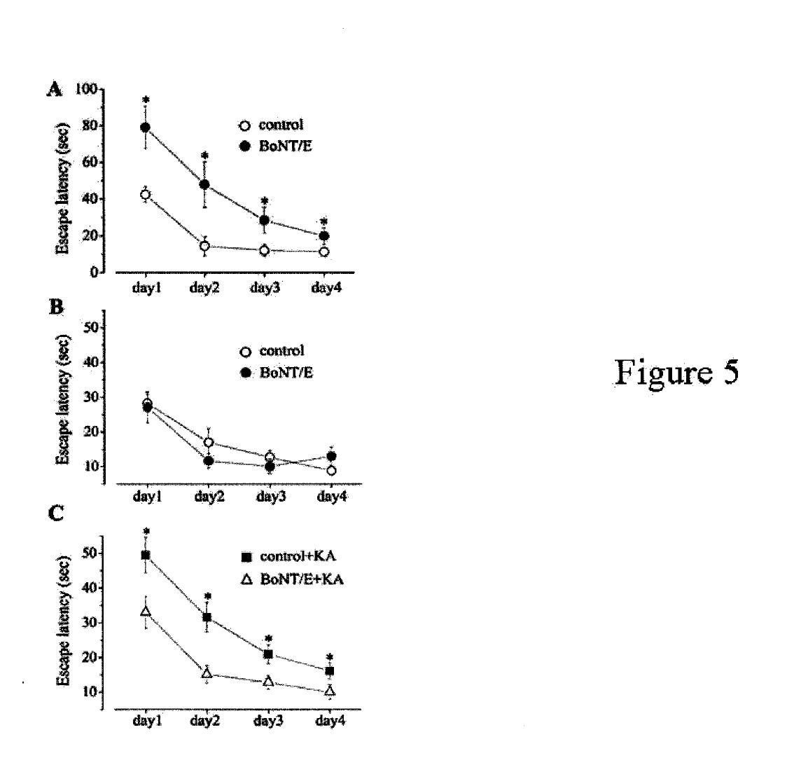

Clear deficits in acquisition of spatial learning were evident in rats tested 3-7 days after BoNT/E, i.e. during the time window of action of the toxin (n=8 animals per group; two-way ANOVA, p<0.01; FIG. 5A). However, in BoNT/E-injected animals (n=5) allowed to recover for 5 weeks, a time at which the effect of BoNT/E is extinguished (see FIG. 1E, F), we found a learning ability indistinguishable from that of control rats (n=10) (two-way ANOVA, p=0.38; FIG. 5B). These long-term BoNT/E-injected rats also showed normal exploratory behavior in the open field (data not shown).

Control+KA rats (black squares, FIG. 5C) performed poorly in the Morris water maze, consistent with previous studies (e.g. Stafstrom et al. (1993) Epilepsia 34:420-432; Mikati et al. (2001) Epilepsy Res 43:97-101). Performance of BoNT/E+KA rats (open triangles, FIG. 5C) was significantly superior to that of control+KA rats (two-way ANOVA, p<0.001). Differences in performance could not be attributed to differences in swim speed, which was similar across the experimental groups (one-way ANOVA, p>0.05; data not shown). Importantly, BoNT/E+KA rats showed spatial learning abilities comparable to that of age-matched naïve rats (two-way ANOVA, p=0.4; compare BoNT/E+KA of FIG. 5C vs. control rats of FIG. 5B).

### Example 5

### Evaluation of the Neuroprotective Effect of BoNT/E Following Seizures

Hippocampal neuronal loss was evaluated in control (n=22) and BoNT/E-infused rats (n=11) treated with KA, at the end of Morris water maze experiments of Example 3 (P77). Rats were perfused with 4% paraformaldehyde and coronal sections throughout the dorsal hippocampus were processed in serial order for immunohistochemistry with mouse anti-NeuN monoclonal antibody (1:500 dilution; Chemicon, Temecula, Calif.). Neuronal damage was scored in areas CA1 and CA3 of the hippocampus according to the following scale (Bozzi et al. (2000) J Neurosci 20:8643-8649; Cilio et al. (2001) Neuropharmacology 40:139-147): 0, no damage; 1, minimal damage (small spots of degeneration); 2, evident loss of pyramidal neurons; 3, complete disruption of hippocampal architecture. An average number of 10 sections per animal were analysed by an investigator unaware of the treatment. A separate score was initially assigned to CA1 and CA3 regions of both sides of each section, and these values were used to calculate the mean damage score for each section. These values were averaged and the obtained damage scores for each animal were plotted. Statistical analysis was performed by the Mann-Whitney rank sum test.

Results:

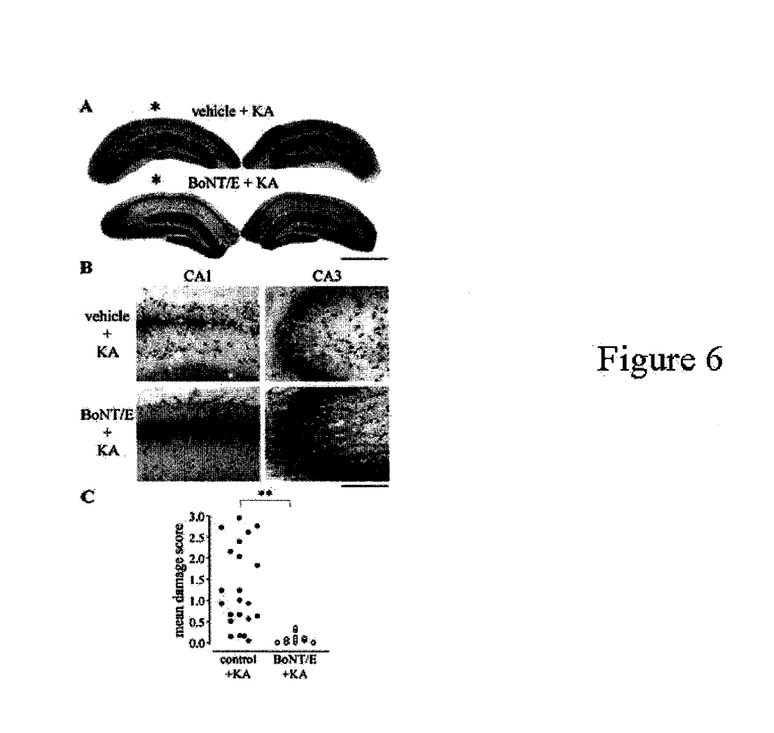

A significant preservation of hippocampal cells was found in BoNT/E-injected rats as compared to abundant neuronal loss in CA1, CA3 and hilus in vehicle-injected rats treated with KA (FIG. 6A-B). Quantification of histological lesions in the dorsal hippocampus of control (black circles; n=22) and BoNT/E-injected (open circles; n=11) rats treated with KA is shown in FIG. 6C.

The animal data reported above clearly indicate that intracerebral delivery of BoNT/E impairs excitatory transmission and has potent anticonvulsant effects on experimental seizures. Remarkably, the effects of BoNT/E are completely reversible and brain function returns to normal when BoNT/E action is off. Based on these properties, a protocol is envisaged in which BoNT/E is used as a diagnostic tool in the evaluation of pharmacoresistant focal epilepsies to predict whether surgical resection will stop seizures.

### Example 6

### Functional Mapping of an Epileptic Focus in Humans Using BoNT/E

A patient with pharmacoresistant focal epilepsy is subjected to a series of diagnostic evaluations to determine the location of the epileptogenic focus. A first step is video-EEG monitoring with scalp electrodes, that is then followed by high-resolution magnetic resonance imaging. MRI is done using standardized protocols (e.g. Francione et al., J. Neurol. Neurosurg. Psychiatry 2003) including T1 and T2 weighted sequences in the three spatial planes. Techniques such as 3T phase array MRI and voxel-based morphometry of gray matter are adopted to increase the yield of identifying subtle morphological alterations that can be correlated with the results from video-EEG monitoring. In cases of negative findings on MRI, positron-emission tomography (PET) can be helpful in identifying the epileptogenic zone. Single photon emission computed tomography (SPECT) scans can also be performed to identify the area of seizure onset. SPECT scans are reconstructed in sagittal, coronal and axial planes (whiting et al. Health Technology Assessment 2006 vol. 10, no. 4).

If seizure origin can be identified using these non-invasive methodologies, stereotactic procedures are employed for the delivery of a solution of BoNT/E to the previously determined epileptogenic focus via an intracerebral cannula. The procedure involves general anesthesia and it involves the following steps:
a) Determining a location of a presumptive epileptogenic focus by means of one or more techniques selected from electroencephalographic techniques, magnetic resonance imaging and tomographic techniques;
b) Injecting a botulinum neurotoxin (BoNT) solution to the presumptive epileptogenic focus in a subject through an intracerebral cannula, the cannula being fixed to an electrode that allows low-frequency bipolar stimulation.
c) Checking the spread of the neurotoxin and the coverage of the presumptive area of seizure onset;
d) Routine checking for occurrence of spontaneous seizures; and
e) Defining the limits of the surgical resection.

The intraparenchymal trajectory of the magnetic resonance-compatible cannula is planned on stereo-angiographic and three-dimensional magnetic resonance images. The cannula is glued to an electrode that allows low-frequency bipolar stimulation. This is an important step for target tissue localization and functional mapping of the explored region. Once precise targeting is confirmed, injection of the BoNT/E solution through the cannula can take place. The injected amount can vary between 0.002×10⁻⁶ and 0.2×10⁻⁶ mg/kg of the affected subject. Injection volume can vary between 0.1 and 10 μl. Small injection volumes (1 μl or less) are preferable since they will result in less diffusion of the neurotoxin, thus making the application more focused to the target area and reducing the risk of undesired effects. Blockade of excitatory transmission and neuronal activity occurs within a few hours after BoNT/E delivery. Spread of the neurotoxin and coverage of the presumptive area of seizure onset is checked via functional neuroimaging. Functional MRI (measuring alterations in blood oxygenation level) is ideal for this purpose as BoNT/E action will produce a localized change in BOLD signal due to blockade of neuronal activity. These effects of BoNT/E persist for at least two weeks and the patient is concurrently checked for the occurrence of spontaneous seizures. If these are prevented within the time window of BoNT/E action, then the silenced area represents the actual epileptogenic focus and the limits of the surgical resection can be accurately selected accordingly.

If the pre-surgical non-invasive data (scalp EEG, MRI, PET and SPECT scans) are discordant or inconclusive in pinpointing the seizure focus, intracranial EEG monitoring may become necessary. Stereotactically introduced intracerebral electrodes (stereo-EEG) are performed to resolve the borders of the epileptogenic zone and to plan an accordingly tailored surgical resection. If the epileptogenic region involves eloquent cortex, either intra-surgical or extra-surgical cortical mapping is recommended.

Such method involves the following steps:

a) implanting a plurality of intracerebral electrodes that allow low-frequency bipolar stimulation in a subject, each one of the electrodes being fixed to an intracerebral cannula;

b) chronic stereo EEG monitoring and recording of spontaneous or induced seizures to define the presumptive epileptogenic zone;

c) intracerebrally injecting, via the cannulas, a solution of a botulinum neurotoxin (BoNT) near the electrode tips located in the area of presumptive seizure onset;

d) post injection chronic stereo EEG monitoring, combined with functional neuroimaging to assess the spread of the neurotoxin effects;

e) confirming or rejecting presumptive localization of the epileptogenic focus on the basis of the ability of the neurotoxin to suppress seizures.

Different kinds of electrodes can be used for the stereo-EEG, depending on brain area that is targeted. Depth electrodes are wires inserted into the brain via a small hole drilled into the skull. Foramen ovale electrodes are wires passed through the foramen ovale to lie under the temporal lobe. Subdural mats are grids of electrodes that are placed over an area of cerebral cortex following a craniotomy. Finally, sphenoidal electrodes are thin wires inserted into the soft tissues anterior to the temporo-mandibular joint using a needle. All electrodes can incorporate a small cannula for intracerebral administration of compounds. All procedures of implantation are performed under general anaesthesia.

Chronic stereo-EEG exploration is done following implantation to record spontaneous seizures in the patient. Seizures can also be induced by electrical stimulation. After a chronic stereo EEG monitoring and recording period (baseline period) of several days, a solution of the neurotoxin is intracerebrally introduced near the electrode tips in the area of presumptive seizure onset. Injection is performed via the cannulas incorporated in the electrodes and results in the blockade of neuronal discharges for at least two weeks. During this period, chronic stereo-EEG monitoring is performed to assess whether spontaneous seizures have been extinguished. The blocked area is identified via functional neuroimaging. Transient blockade of seizure activity by BoNT/E allows unambiguous identification of the epileptogenic focus.

### Example 7

### Intracranial Administration of Other BoNT Serotypes for Functional Mapping of the Epileptogenic Focus

The same detailed procedure described in Example 6 can be conceivably performed with other botulinum neurotoxin serotypes, namely the A, B, C, D, F and G serotypes. The technique consists of baseline EEG monitoring and neuroimaging in a patient with focal intractable epilepsy, followed by intracranial delivery of the neurotoxin to the presumptive epileptogenic focus via an implanted cannula. Absence of spontaneous recurrent seizures following the application of the method is combined with visualization of the silenced area by functional MRI or other functional imaging methods. This allows functional mapping of the focus and accurate prediction of the surgical outcome.

Use of other BoNT serotypes may be useful when different durations of the anti-ictal effect are desired. For example, BoNT/A cleaves the same substrate as BoNT/E, namely SNAP-25, but exhibits longer duration of action. Persistence of BoNT/A effects may ensure a prolonged monitoring of spontaneous seizures following the application of the method.

To conclude, the Examples reported above demonstrate that intracerebral administration of BoNT serotypes produces a reversible blockade of transmitter release and synaptic activity in the targeted brain area. This results in a powerfill anticonvulsant action on both generalized and focal seizures. Indeed, the EEG data demonstrate that BoNT administration delays the onset and decreases the number and duration of EEG ictal episodes after focal delivery of KA. The behavioral analysis of seizures after systemic injection of KA shows that limbic status epilepticus is prevented by BoNT injection. The reversibility of BoNT effects and their anti-ictal properties make them effective diagnostic tools in pre-surgical evaluation of epileptic patients. Mapping of the area of seizure onset is achieved via temporary inactivation of the presumptive epileptogenic focus with intracranial BoNT injection. Cessation of spontaneous ictal events in the follow-up (as determined at the EEG and/or clinical level) is combined with imaging of the silenced area and provides a powerful diagnostic method for predicting if surgical resection will be effective in curing the disease.

The specification is most thoroughly understood in the light of the teachings of the references cited within the specification. The aspects within the specification provide an illustration of aspects of the invention and should not be construed to limit the scope of the invention. The skilled artisan readily recognizes that many other aspects are encompassed by the invention. All publications cited in this disclosure are incorporated by reference in their entirety. To the extent the material incorporated by reference contradicts or is inconsistent with the present specification, the present specification will supercede any such material. The citation of any references herein is not an admission that such references are prior art to the present invention.

Unless otherwise indicated, all numbers expressing quantities of substances, application conditions and so forth used in the specification, including claims, are to be understood as being modified in all instances by the term “about”. Accordingly, unless otherwise indicated to the contrary, the numerical parameters are approximations and may vary depending upon the desired properties sought to be obtained by the present invention. Those skilled in the art will recognize, or be able to ascertain using no more than routine experimentation, many equivalents to the specific aspects of the invention described herein. Such equivalents are intended to be encompassed by the following claims:

1. A method for functionally identifying an epileptogenic focus in pre-surgical evaluation in affected subjects with intractable epilepsy, the method comprising the delivery of an effective dose of a botulinum neurotoxin (BoNT) to a presumptive epileptogenic focus in the disease-compromised central nervous system of a mammal, under conditions whereby said effective dose of said botulinum neurotoxin interacts with the soluble N-ethylmaleimide-sensitive factor-attachment receptor (SNARE) proteins, thus impairing neurotransmission.

2. The method of claim 1, wherein said botulinum neurotoxin is botulinum neurotoxin serotype E (BoNT/E)

3. The method of claim 2, wherein said effective dose is comprised between 0.002×10⁻⁶ and 0.2×10⁻⁶ mg/kg of the affected subject.

4. The method of claim 1, wherein said disease is any type of epileptic seizure condition.

5. The method of claim 1, wherein said presumptive epileptogenic focus is located in a region of the CNS selected from the group consisting of: the cerebral cortex, the frontal lobe, the parietal lobe, the temporal lobe, the occipital lobe, the striatum, the hippocampus, the amygdala, the thalamus, the hypothalamus, the mesencephalon, the cerebellum, the brainstem, the pons, the medulla, the spinal cord.

6. The method of claim 1, wherein said presumptive epileptogenic focus is located in the hippocampus or the cerebral cortex of the brain of said mammal.

7. The method of claim 1, wherein said delivery of said effective dose of the botulinum neurotoxin is performed either by acute stereotaxic microinjection or by infusion through previously implanted cannulas.

8. The method of claim 1, wherein the total volume of the said effective dose ranges between 0.1 and 10 μl.

9. A method for functionally identifying an epileptogenic focus in pre-surgical evaluation in affected subjects with intractable epilepsy, the method comprising the steps of:
a) Determining a location of a presumptive epileptogenic focus by means of one or more techniques selected from electroencephalographic techniques, magnetic resonance imaging and tomographic techniques.
b) Injecting a botulinum neurotoxin (BoNT) solution to said presumptive epileptogenic focus in a subject through an intracerebral cannula, said cannula being fixed to an electrode that allows low-frequency bipolar stimulation;
c) Checking the spread of said neurotoxin and the coverage of said presumptive area of seizure onset;
d) Routine checking for occurrence of spontaneous seizures; and
e) Defining the limits of surgical resection.

10. Method of claim 9, wherein said cannula is a magnetic resonance-compatible cannula and its intraparenchymal trajectory is planned on stereo-angiographic and three-dimensional magnetic resonance images.

11. Method of claim 9, wherein said neurotoxin is injected in an amount that varies between 0.002×10⁻⁶ and 0.2×10⁻⁶ mg/kg of the affected subject.

12. Method of claim 9, wherein said neurotoxin is injected in an amount that varies between 0.01×10⁻⁶ and 0.15×10⁻⁶ mg/kg of the affected subject.

13. Method of claim 11, wherein said neurotoxin solution is injected in a volume varying between 0.1 and 10 μl.

14. Method of claim 11, wherein said neurotoxin solution is injected in a volume varying between 0.1 and 1 μl.

15. Method of claim 9, wherein said spread of the neurotoxin and said coverage of the presumptive area of seizure onset are checked via functional neuroimaging over a period of time of 1 to 15 days after neurotoxin injection.

16. Method of claim 9, wherein said spread of the neurotoxin and said coverage of the presumptive area of seizure onset are checked via functional neuroimaging over a period of time of 3 to 10 days after neurotoxin injection.

17. Method of claim 9, wherein said routine checking is carried out over a period of time of 1 to 15 days after neurotoxin injection.

18. Method of claim 9, wherein said routine checking is carried out over a period of time of 3 to 10 days after neurotoxin injection.

19. A method for functionally identifying an epileptogenic focus in pre-surgical evaluation in affected subjects with intractable epilepsy, the method comprising the steps of:
a) Implanting a plurality of intracerebral electrodes that allow low-frequency bipolar stimulation in a subject, each one of said electrodes being fixed to an intracerebral cannula;
b) Chronic stereo EEG monitoring and recording of spontaneous or induced seizures to define the presumptive epileptogenic zone;
c) Intracerebrally injecting, via said cannulas, a solution of a botulinum neurotoxin (BoNT) near the electrode tips located in the area of presumptive seizure onset;
d) Post injection chronic stereo EEG monitoring, combined with functional neuroimaging to assess the spread of the neurotoxin effects;
e) Confirming or rejecting presumptive localization of the epileptogenic focus on the basis of the ability of the neurotoxin to suppress seizures.

20. Method of claim 19, wherein said intracerebral electrodes are selected from the group comprising: depth electrodes; foramen ovate electrodes; subdural mats; and sphenoidal electrodes.

21. Method of claim 19, wherein said neurotoxin is injected in an amount that varies between 0.002×10⁻⁶ and 0.2×10⁻⁶ mg/kg of the affected subject.

22. Method of claim 19, wherein said neurotoxin is injected in an amount that varies between 0.0×10⁻⁶ and 0.15×10⁻⁶ mg/kg of the affected subject.

23. Method of claim 21, wherein said neurotoxin solution is injected in a volume varying between 0.1 and 10 μl.

24. Method of claim 21, wherein said neurotoxin solution is injected in a volume varying between 0.1 and 1 μl.

25. Method of claim 19, wherein said seizures are spontaneous or induced by electrical stimulation.

26. Method of claim 19, wherein said step d) consists of identifying the blocked area by functional neuroimaging.

27. Method of claim 19, wherein said steps b) and d) of chronic stereo EEG monitoring is carried out over a period of time of 1 to 15 days.

28. Method of claim 19, wherein said steps b) and d) of chronic stereo EEG monitoring is carried out over a period of time of 3 to 10 days.
